(19) United States
(12) Patent Application Publication
(10) Pub. No.: US 2005/0124038 A1
(43) Pub. Date: Jun. 9, 2005

(54) Transfection of cartilage and matrices thereof in vitro

(75) Inventors: Dean J. Aguiar, Saint Peters, MO (US); Adam P. Skepner, Chesterfield, MO (US); H. Davis Adkisson IV, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignees: Pharmacia Corporation; ISTO Technologies, Inc.

(21) Appl. No.: 10/891,986

(22) Filed: Jul. 14, 2004

Related U.S. Application Data
(60) Provisional application No. 60/486,990, filed on Jul. 14, 2003.

(51) Int. Cl. C07H 21/04; C12N 15/861

(52) U.S. Cl. 435/69.1; 435/455; 435/456; 435/325; 530/356; 536/23.5

(57) ABSTRACT

The invention provides methods for transfecting and/or transducing neocartilage or juvenile cartilage comprising infecting cultured neocartilage or juvenile cartilage with a recombinant vector. Preferably, the vector is adenovirus fiber type 51. Transfected and/or transduced chondrocyte cells and intact neocartilage or juvenile cartilage are also provided.

### CROSS-REFERENCES TO RELATED APPLICATIONS

This application claims priority from Provisional Application Ser. No. 60/486,990 filed on Jul. 14, 2003, which is incorporated herein by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISC

Not applicable.

### BACKGROUND OF THE INVENTION

1. Field of the Invention

The invention relates to transfection of cartilage and matrices thereof in vitro, particularly transfection of neocartilage, juvenile cartilage and matrices thereof using adenoviral vectors.

2. Description of the Related Art

Transfecting mammalian cells is a common technique that is routinely used in biomedical and genetic applications. Transfection refers to a method of introducing nucleic acid material into a target cell in a non-lethal manner. Once transfection is used to introduce a nucleic acid into a cell, the nucleic acid may direct synthesis of new RNA and/or new proteins. These RNA and/or proteins may provide new functionality for the cell or suppress the expression of other genes. Mechanisms of action of potential therapeutic targets may be elucidated by transfecting a series or array of related polynucleotides into different cell populations with proper controls.

Gene suppression, on the other hand, may occur by transfecting a gene which encodes a suppressor protein or an antisense nucleic acid construct. A suppressor protein is any protein that reduces or eliminates the expression of another gene. An antisense nucleic acid is a nucleic acid that is complementary to an expressed gene. The complementary nucleic acid may hybridize to the sense RNA of the targeted gene to form a non-functional double stranded RNA which is not translated into protein. In any case, the expression (i.e., translation) of the targeted gene is suppressed by expression (i.e., transcription) of antisense DNA.

Numerous techniques have been developed for transfection of cells in vitro. The basic goal of all transfection techniques is to introduce the nucleic acid into a target cell. Transfection techniques may be broadly classified as either direct or indirect methods. Direct methods involve the manual introduction of nucleic acid. Examples of direct transfection include microinjection or microprojectile transfection. Indirect transfection techniques are numerous but can be broadly classified into viral transfection techniques, liposome transfection techniques and phagocytosis techniques.

Direct transfection can be performed by microinjection, microprojectile transfection or by electrotransfection or laser transfection. Microinjection involves the manual injection of nucleic acid solutions into a cell by the use of a small needle (usually a drawn glass capillary) under a microscope. Microprojectile transfection involves the coating small particles with nucleic acid and shooting the particles into a cell with a high velocity gun. Laser transfection or electrotransfection involves puncturing a temporary hole in the cell membrane and allowing nucleic acid in the surrounding media to enter.

A disadvantage of current direct transfection techniques is that the procedures are only effective on cells and tissue parts that can be exposed and accessible to the microinjection needle or microprojectile gun. Thus, the interior of tissues, such as cartilage tissue, are difficult to transfect without partial removal of extracellular matrices formed by chondrocyte cells making up the cartilage. In addition, direct transfection would be inefficient and ineffective when used to transfect the cells of an organ or tissue having thousands or hundreds of thousands of cells requiring the integration of a gene.

The major disadvantage of indirect transfection is low efficiency. One type of indirect transfection uses viruses to introduce nucleic acid into cells. Many viruses used for transfection purposes include SV40, polyoma, Type 5 adenovirus, Epstein-Barr, vaccinia, herpes simplex, and retrovirus. These viruses have traditionally suffered from low transfection efficiency especially when used with cartilage tissue. Other indirect transfection methods such as liposome transfection and DNA-calcium phosphate transfection also suffer from low transfection efficiency when used with cartilage tissue.

Therefore, what is needed is a new method of transfecting cartilage tissue in vitro which enhances the transfection efficiency for targeted gene integration.

### BRIEF SUMMARY OF THE INVENTION

Accordingly, it is an object of the invention to overcome these and other problems associated with the related art. These and other objects, features and technical advantages are achieved by transfecting cartilage with recombinant vectors, including adenovirus fiber type 51, which surprisingly enhances cartilage transfection efficiency by about one magnitude.

This invention provides a method for transfecting neocartilage tissue or a cartilage matrix to confer on said tissue or matrix the ability to express a desired protein, the method comprising infecting cultured neocartilage tissue or a cartilage matrix with a recombinant vector, said recombinant vector comprising a polynucleotide encoding said protein.

In accordance with a further aspect of the invention, the recombinant vector further comprises a promoter sequence operably linked to said polynucleotide encoding said protein, said promoter sequence capable of driving the expression of said polynucleotide in said neocartilage tissue or cartilage matrix. Preferably, said polynucleotide further encodes a selectable marker protein operably linked to a second promoter sequence. In another aspect, the vector is selected from the group consisting of an adenovirus, adeno-associated virus and retrovirus. Preferably, the adenovirus is adenovirus fiber type 51.

In accordance with yet another aspect of the invention, said neocartilage tissue or a cartilage matrix is mammalian neocartilage tissue or a mammalian cartilage matrix. More preferably, said mammalian neocartilage tissue or mammalian cartilage matrix is a human neocartilage tissue or a human cartilage matrix.

In accordance with yet another aspect of the invention, the cartilage matrix comprises a cartilage matrix of neocartilage or juvenile cartilage particles, or a combination thereof; and a biocompatible chondro-conductive/inductive matrix. In one aspect, the cartilage matrix may comprise particles of neocartilage. In another aspect, the cartilage matrix may comprise particles of juvenile cartilage. In another aspect, the biocompatible chondro-conductive/inductive matrix is selected from the group consisting of fibrinogen, fibrinogen/thrombin, albumin, in-situ forming PEG hydrogel, fibrin/hyaluronate, fibrin/collagen/hyaluronate, PEG/hyaluronate, PEG/collagen, other plasma and protein-based adhesives and sealants, other natural adhesives and sealants and any combination thereof.

In yet another aspect, the biocompatible chondro-conductive/inductive matrix further comprises an osteo-conductive matrix. Preferably, at least about 5%, and more preferably 10%, of chondrocytes comprising the neocartilage tissue or cartilage matrix are conferred the ability to express the desired protein.

In accordance with yet another aspect of the invention, an isolated chondrocyte cell is provided comprising a recombinant adenovirus vector comprising a polynucleotide encoding a protein operably linked to a promoter sequence, wherein said promoter sequence drives the expression of said polynucleotide in said isolated chondrocyte cell. In a further aspect of the invention, a composition of matter is provided comprising intact neocartilage tissue or a cartilage matrix, said intact neocartilage tissue or cartilage matrix comprising a recombinant adenovirus vector further comprising a polynucleotide encoding a protein operably linked to a promoter sequence, wherein said promoter sequence drives the expression of said polynucleotide in said intact neocartilage tissue or cartilage matrix.

These and other features, aspects and advantages of the present invention will become better understood with reference to the following description, examples and appended claims.

### BRIEF DESCRIPTION OF THE SEVERAL VIEWS OF THE DRAWINGS

FIG. 1. Safarin-O staining for proteoglycan content in neocartilage 72 hours post-transduction with (a) control non-transduced, (b) LacZ, (c) MMP-13, and (d) ADAM-TS4.

FIG. 2. MMP-13 expression and activity in conditioned media from neocartilage 48 hours post-transduction with MMP-13.

FIG. 3. The addition of 5 ng/ml MMP-9 protein to MMP-13 transduced neocartilage resulted in the release of type II collagen neoepitope at 48 hours post-transduction with virus.

### DETAILED DESCRIPTION OF THE INVENTION

Abbreviations and Definitions

To facilitate understanding of the invention, a number of terms and abbreviations as used herein are defined below as follows:

The term “juvenile cartilage” refers to a chondrocyte cell, cells or cartilage tissue that is committed to become articular cartilage but is still capable of undergoing growth, differentiation and maturation. In general, such chondrocytes are found in tissue from prepubescent individuals. In humans, preferably chondrocytes are from those less than fifteen years of age, and more preferably, less than two years of age. Typically, immature or juvenile chondrocytes express an enhanced ability to synthesize and organize a hyaline cartilage extra-cellular matrix. This activity usually is highest in cells freshly isolated from donor tissue and falls off rapidly during subsequent manipulation such as passage and expansion.

The term “neocartilage” refers to cartilage characterized by one or more of the following attributes: containing membrane phospholipids enriched in Mead acid, containing membrane phospholipids depleted in linoleic or arachidonic acid, being substantially free of endothelial, bone and/or synovial cells, having a S-GAG content of at least about 400 mg/mg, positive for type II collagen expression, being substantially free of type I, III and X collagen, containing a matrix substantially free of biglycan, having multiple layers of cells randomly arranged, rather than separated into distinct zones of chondrocyte maturation, being enriched in high molecular weight aggrecan, being produced in vitro being essentially free of non-cartilage material, or being characterized by having multiple layers of cells surrounded by a substantially continuous insoluble glycosaminoglycan and collagen-enriched hyaline extracellular matrix.

The term “biocompatible” refers to materials which, when incorporated into the invention, have low toxicity, acceptable foreign body reactions in the living body, and acceptable affinity with living tissues.

The term “chondro-inductive” refers to the ability of a material to induce the production of articular cartilage from chondroprogenitor cells, juvenile cartilage or neocartilage. A chondro-inductive/conductive material may act directly as a growth factor which interacts with precursor cells to induce the chondrocyte differentiation and/or maturation, or the material may act indirectly by inducing the production of other chondro-inductive/conductive factors, such as growth factors. This induction may optionally include signaling, modulating, and transforming molecules.

The term “chondro-conductive” refers to materials which provide an environment for ingrowth and orientation of chondrocyte cells or chondroprogenitor cells from surrounding tissues.

The term “chondro-inductive/conductive” refers to the characteristic of being either chondro-inductive or chondro-conductive, or optionally both.

The term “matrix” refers to surrounding substance(s) within which something is contained.

The term “osteo-conductive” refers to materials which provide an environment for ingrowth and orientation of osteogenic cells from surrounding tissues.

Transfection of Cartilage and Matrices Thereof In Vitro

The present invention provides methods for transfecting neocartilage tissue or a cartilage matrix of neocartilage or juvenile cartilage particles, or a combination thereof and a biocompatible chondro-conductive/inductive matrix to confer on said tissue or matrix the ability to express a desired protein. Some embodiments comprise infecting cultured neocartilage tissue with a recombinant vector which further comprises a polynucleotide encoding the protein.

In one embodiment, the methods of the present invention may use in vitro a 3-dimensional, hyaline-like neocartilage. Such neocartilage has biochemical and morphologic properties reflective of juvenile cartilage-like tissue and can be grown from juvenile human chondrocytes (see U.S. Pat. Nos. 6,235,316 and 6,645,764, incorporated herein by reference in its entirety). This tissue is composed of type II collagen and proteoglycans, but does not contain type I and X collagens. The neocartilage tissue may also be of other mammalian species including farm animals (e.g., horses, goats, cows, pigs, sheep) and household pets (e.g., dogs, cats). Neocartilage may also be provided for avians, fish and other species which are valuable in models to study transfection of neocartilage in vitro. Most preferably, the mammalian neocartilage tissue is human neocartilage tissue.

In a related embodiment, the methods of the present invention may also use a cartilage matrix of neocartilage or juvenile cartilage particles, or a combination thereof and a biocompatible chondro-conductive/inductive matrix. Some embodiments may further comprise an osteo-conductive matrix. The cartilage matrix may be distributed throughout substantially all of the biocompatible chondro-conductive matrix or just a portion of the matrix, the portion may range from 90 to 10%. In some embodiments the surface-to-volume ratio of the cartilage particles is greater than 1. In any embodiment the biocompatible chondro-conductive/inductive matrix may be fibrinogen, fibrinogen/thrombin, albumin, in-situ forming PEG hydrogel, fibrin/hyaluronate, fibrin/collagen/hyaluronate, PEG/hyaluronate, PEG/collagen, other plasma and protein-based adhesives and sealants, other natural adhesives and sealants and any combination thereof. In any embodiment the composition may further comprise an osteo-conductive matrix. The osteo-conductive matrix may be fibrinogen, fibrinogen/thrombin, fibrin/tri-calcium phosphate, fibrin/collagen/tri-calcium phosphate, fibrin/hyaluronate/tri-calcium phosphate, in-situ forming PEG hydrogel sealants in-situ forming PEG hydrogel sealants, PEG/tri-calcium phosphate, PEG/collagen, demineralized bone matrix, and any combination thereof.

The biocompatible chondro-conductive/inductive matrix may comprise any appropriate compound or combination of compounds that is inductive or conductive for the formation or repair of articular cartilage in the compositions and methods. The chondro-conductive/inductive matrix may comprise fibrinogen. The fibrinogen may be from any suitable source. For example, one skilled in the art will recognize that fibrinogen may be derived from blood bank products—either heterologous (pooled or single-donor) or autologous cryoprecipitate or fresh frozen plasma. Fibrinogen can also be derived from autologous fresh or platelet-rich plasma, obtained using cell-saver or other techniques. U.S. Pat. No. 5,834,420 also discloses a method for obtaining fibrinogen.

In other embodiments the biocompatible chondro-conductive/inductive matrix comprises thrombin. The thrombin may be from any suitable source. One skilled in the art will recognize that thrombin can be isolated by well known means or purchased commercially. See U.S. Pat. No. 4,965,203, and Berliner, J L, Thrombin: Structure and Function (Ed) Plenum Pub Corp; (1992) for exemplary methods of isolation and/or purification. In any embodiment the biocompatible chondro-conductive/inductive matrix may comprise a combination of fibrinogen and thrombin. The chondro-conductive/inductive matrix may contain equal proportions of fibrinogen and thrombin or more of either fibrinogen than thrombin or more thrombin than fibrinogen. When used in combination the two may be in any proportion, ranging from one part of either compared to the amount of the other up to equal proportions of each of the two.

Regardless of whether the fibrinogen and/or the thrombin are part of the cartilage matrix or part of the biocompatible chondro-conductive/inductive matrix, when practicing any embodiment of the invention the fibrinogen and thrombin components should be kept separate prior to the time of use. The fibrinogen and the thrombin are then brought into contact with each other at the time of use. A common type of applicator that may be used for this purpose consists of a double syringe, joined by a Y-connector where the components mix as they emerge. This type of applicator, used with a blunt cannula, is useful for combining the thrombin and the fibrinogen and also useful in the methods of the invention for disposing or transplanting the inventive compositions to a site wherein articular cartilage repair is desired. In cases where the articular cartilage repair site is open for repair, the fibrinogen and/or thrombin can also be used with a spray attachment to cover surfaces; or the fibrinogen and/or thrombin may be applied to an absorbable carrier or dressing, such as a cellulose sponge, collagen fleece, vital or devitalized periosteum or any other suitable means.

In various embodiments the chondro-conductive/inductive matrix may comprise one or more of fibrinogen, thrombin, fibrinogen/thrombin (Tisseel), albumin, in-situ forming PEG hydrogel, fibrin, hyaluronate, fibrin/hyaluronate, collagen hyaluronate, fibrin/collagen/hyaluronate, PEG/hyaluronate, PEG/collagen, PEG base sealants (CoSeal), or other plasma and protein-based adhesives and/or sealants, other natural adhesives and/or sealants and combinations thereof, that are biocompatible with regard to the articular cartilage repair or replacement and are inductive or conductive for the cartilage matrix in the repair or replacement of articular cartilage. The matrices may also include materials which are not yet known, but which provide characteristics relating to these components which are similar to the materials described herein.

The cartilage matrix in any embodiment may comprise neocartilage or juvenile cartilage or a combination of juvenile cartilage and neocartilage. The neocartilage and juvenile cartilage may be in any proportion to each other, ranging from one cell or part of either compared to the other up to equal proportions of each of the two. For example the cartilage matrix may contain equal proportions of neocartilage and juvenile cartilage or more of either neocartilage than juvenile cartilage or more juvenile cartilage than neocartilage. In some embodiments the compositions of the invention further comprise a particulate osteo-conductive matrix. The juvenile or neocartilage is in the form of particles in the cartilage matrix. The particles increase the surface to volume ratio in the cartilage matrix, which allows for improved integration and metabolite and growth factor exchange, which advantageously results in enhanced viability and shelf life for the compositions. The juvenile and neocartilage particles in the cartilage matrix may vary in size from single cells with associated matrix to 100 mm³ in size depending on application or defect type. For a somewhat typical defect of 2 cm, at least 1×10⁶ to 2×10⁶ cells would be disposed, preferably 2×10⁶ to 4×10⁶, and most preferably 10×10⁶ to 20×10⁶. The amount of cells used would vary depending on the specific circumstances of a defect in need of repair and the goals of the patient. For example, one skilled in the art would recognize that on average, adult tissue has about a 5 to 10% cell count per gram of tissue. This equates to about a 7% fill. However, some cell death will likely occur during maturation so a higher initial cell count is preferable.

In terms of providing economic ratios of tissue to % fill of defects, to maximize tissue use, approximately 300 mg of tissue would provide for about a 50% defect fill, although less, approximately 200 mg, for a 30% defect fill, and most preferably, for a 10% defect fill, 60 mg would be utilized.

The matrix portion of the cartilage matrix may comprise thrombin, fibrinogen, media or fibrinogen in combination with media or thrombin in combination with media. Any suitable media may be used for the media component. Examples of suitable media include, but are not limited to a conditioned growth medium adapted for use in growing cartilage cell cultures which contains heparin-binding growth factors, at least one of which is a cartilage-derived morphogenetic protein (Chang et al., J. Biol Chem 269: 28227-28234), other pre-conditioned medias, Dulbecco's modified Eagle's medium (DMEM), Minimum Essential Medium and RPMI (Roswell Park Memorial Institute) medium. The culture medium may also comprise ascorbate, and/or exogenous autocrine growth factors.

The juvenile cartilage in the invention may be from any suitable source. The juvenile cartilage or chondrocytes used in the composition may be harvested from donor tissue and prepared by dividing or mincing the donor cartilage into small pieces or particles. The juvenile cartilage particles may comprise juvenile cells or tissue, which may be intact, minced or disrupted, such as by homogenizing the tissue. Examples of sources of donor cartilage include autologous or allogenic sources. In the case of autologous grafts, cartilage is harvested from cartilaginous tissue of the patient's own body. Typical sources for autologous donor cartilage include the articular joint surfaces, intercostals cartilage, and cartilage from the ear or nasal septum. In the case of allografts, the cartilage may be taken from any appropriate non-identical donor, for example from a cadaveric source, other individuals or a transgenic source or similar appropriate source.

In any embodiment of the invention the cartilage matrix may comprise juvenile cartilage (without neocartilage) in any suitable tissue culture media. The juvenile cartilage may also comprise juvenile cartilage tissue in a matrix of thrombin or juvenile cartilage in a matrix of fibrinogen. In any embodiment that includes neocartilage in the matrix, the cartilage matrix may comprise neocartilage cells in any suitable tissue culture media. The neocartilage matrix may also comprise neocartilage in a thrombin matrix or neocartilage in a fibrinogen matrix.

In embodiments having neocartilage, the neocartilage may be from any suitable source. The neocartilage particles may comprise neocartilage cells or tissue, which may be intact, minced or disrupted, such as by homogenizing the tissue. The neocartilage may be either autologous or allogenic. Examples of suitable sources include commercially available sources, such as Carticel® (Genzyme Biosurgery, Cambridge, Mass.), embryonic sources, tissue culture sources or any other suitable source. For example a cell culture may be produced to grow neocartilage by isolating immature chondrocytes, e.g., fetal, neonatal, and pre-adolescent chondrocytes from donor articular cartilage. The neocartilage of the inventive cartilage matrix may be obtained by culturing chondrocytes under suitable culture conditions known in the art, such as growing the cell culture at 37° C. in a humidified atmosphere with the addition of 2-10% carbon dioxide, preferably 5%. Chondrocytes may be isolated by methods known in the art such as by sequential enzyme digestion techniques. The isolated chondrocytes may then be seeded directly on a tissue culture vessel in any suitable media. Also see, for examples of other sources, U.S. Pat. No. 5,326,357 which describes methods to produce a continuous cartilaginous tissue and U.S. Pat. No. 6,235,316 which discloses neocartilage compositions and uses, which are incorporated by reference, herein in their entirety.

The juvenile or neocartilage tissue for the cartilage matrix can be from any vertebrate organism, including mammalian, avian and fish replacement tissue, most preferably from the same species as the recipient, for example human donor tissue for human replacement and equine tissue for equine use. Furthermore, mammalian replacement tissue can be produced using chondrocytes from transgenic animals which may have been genetically engineered to prevent immune-mediated xenograft rejection.

In embodiments where the matrix portion of the cartilage matrix comprises tissue culture media, without fibrinogen or thrombin, then the biocompatible chondro-conductive/inductive matrix preferably comprises fibrinogen and thrombin. In embodiments where the matrix portion of the cartilage matrix comprises media and fibrinogen, then the biocompatible chondro-conductive/inductive matrix preferably comprises thrombin. In embodiments where the matrix portion of the cartilage matrix comprises media and thrombin, then the biocompatible chondro-conductive/inductive matrix preferably comprises fibrinogen.

In different embodiments various combinations of the cartilage matrix and the biocompatible chondro-conductive/inductive matrix are possible. By way of non-limiting example, an embodied composition may comprise juvenile cartilage and thrombin in the cartilage matrix with the biocompatible chondro-conductive/inductive matrix comprising media and fibrinogen.

In another embodiment the cartilage matrix may comprise neocartilage and thrombin with the biocompatible chondro-conductive/inductive matrix comprising media and fibrinogen. In another embodiment the cartilage matrix may comprise a combination of juvenile and neocartilage in thrombin with the biocompatible chondro-conductive/inductive matrix comprising media and fibrinogen.

In any embodiment the compositions may further comprise an osteo-conductive matrix. The osteo-conductive matrix comprises bone particles. The bone particles may be from any suitable source. The osteo-conductive matrix may include but not be limited to fibrinogen/thrombin (Tisseel), fibrin/tri-calcium phosphate, fibrin/collagen/tri-calcium phosphate, fibrin/hyaluronate/tri-calcium phosphate PEG base sealants (CoSeal), PEG/tri-calcium phosphate, PEG/collagen (FibroGen) and any of the above components mixed with demineralized bone matrix. The osteo-conductive matrix may be purchased from commercial sources, such as the demineralized bone matrix compositions Grafton® (Osteotech, Eatontown, N.J.). Examples of other sources suitable for the osteo-conductive matrix include those disclosed in U.S. Pat. No. 5,356,629, U.S. Pat. No. 6,437,018 and U.S. Pat. No. 6,327,257. Suitable compositions may comprise demineralized bone, demineralized bone matrix, nondecalcified bone, cancellous bone or combinations of the same and a gel material. The osteo-conductive matrix may also comprise a porous solid, semisolid, paste or gel material including materials such as gelatin, hyaluronic acid, collagen, amylopectin, demineralized bone matrix, and/or calcium carbonate fibrinogen/thrombin, fibrin/tri-calcium phosphate, fibrin/collagen/tri-calcium phosphate, fibrin/hyaluronate/tri-calcium phosphate, in-situ forming PEG hydrogel sealants in-situ forming PEG hydrogel sealants, PEG/tri-calcium phosphate, PEG/collagen, demineralized bone matrix, and any combination thereof.

Osteoconductive materials are generally porous materials and are able to provide latticework structures such as the structure of cancellous bone or similar to cancellous bone. Such materials may generally facilitate blood-vessel incursion and new bone formation into a defined passive trellis-like support structure, as well as potentially supporting the attachment of new osteoblasts and osteoprogenitor cells. Osteoconductive materials may provide an interconnected structure through which new cells can migrate and new vessels can form.

Examples of materials suitable for the osteoconductive matrix include those disclosed in U.S. Pat. No. 5,356,629 which discloses a composition of polymethylacrylate biocompatible particles dispersed in a matrix of cellulose ether, collagen or hyaluronic acid and U.S. Pat. No. 6,437,018 which includes a composition of demineralized bone matrix (DBM) in an aqueous carrier that is sodium hyaluronate in a phosphate buffered aqueous solution. U.S. Pat. No. 6,327,257 discloses compositions with demineralized bone, nondecalcified bone, cancellous bone and a gel material. There are also compositions that are available commercially, including demineralized bone matrix compositions such as Grafton® (Osteotech, Eatontown, N.J.). These compositions typically comprise a porous solid, semisolid, paste or gel material including materials such as gelatin, hyaluronic acid, collagen, amylopectin, demineralized bone matrix, and/or calcium carbonate, to create an osteoconductive environment. The compositions also often include osteoinductive factors, as are known in the art.

In some embodiments the composition optionally further comprises other components or compounds to address the needs of a particular articular cartilage injury or circumstance or a specific patient's individual needs. By way of non-limiting example the biocompatible chondro-conductive/inductive matrix may in some instances comprise albumin, in-situ forming PEG hydrogel, fibrin/hyaluronate, fibrin/collagen/hyaluronate, PEG/hyaluronate, PEG/collagen, other plasma and protein-based adhesives and sealants, other natural adhesives and sealants and any combination of these.

In any embodiment the cartilage matrix may be distributed throughout substantially all of the biocompatible chondro-conductive/inductive matrix. Alternatively the cartilage matrix may be distributed throughout a portion of the biocompatible chondro-conductive/inductive matrix. The cartilage matrix may be distributed throughout 90% or less of the biocompatible chondro-conductive/inductive matrix. The cartilage matrix may also be distributed throughout 80% or less of the biocompatible chondro-conductive/inductive matrix. The cartilage matrix may also be distributed throughout 70% or less of the biocompatible chondro-conductive/inductive matrix. The cartilage matrix may also be distributed throughout 60% or less of the biocompatible chondro-conductive/inductive matrix. The cartilage matrix may also be distributed throughout 50% or less of the biocompatible chondro-conductive/inductive matrix. The cartilage matrix may also be distributed throughout 40% or less of the biocompatible chondro-conductive/inductive matrix. The cartilage matrix may also be distributed throughout 30% or less of the biocompatible chondro-conductive/inductive matrix. The cartilage matrix may also be distributed throughout 20% or less of the biocompatible chondro-conductive/inductive matrix. The cartilage matrix may also be distributed throughout 10% or less of the biocompatible chondro-conductive/inductive matrix.

Similarly, in embodiments where the compositions and methods further comprise an osteo-conductive matrix, the osteo-conductive matrix may be distributed throughout substantially all of the composition. Alternatively the osteo-conductive matrix may be distributed throughout a portion of the composition. It may be desirable in some embodiments to have the osteo-conductive matrix disposed to contact bone in a defect that has involvement of both bone and articular cartilage. The osteo-conductive matrix may be distributed throughout 90% or less of the composition. The osteo-conductive matrix may also be distributed throughout 80% or less of the composition. The osteo-conductive matrix may also be distributed throughout 70% or less of the composition. The osteo-conductive matrix may also be distributed throughout 60% or less of the composition. The osteo-conductive matrix may also be distributed throughout 50% or less of the composition. The osteo-conductive matrix may also be distributed throughout 40% or less of the composition. The osteo-conductive matrix may also be distributed throughout 30% or less of the composition. The osteo-conductive matrix may also be distributed throughout 20% or less of the composition. The osteo-conductive matrix may also be distributed throughout 10% or less of the composition.

Unlike transfection of human articular cartilage, transfection of neocartilage tissue or a cartilage matrix of neocartilage or juvenile cartilage particles, or a combination thereof and a biocompatible chondro-conductive/inductive matrix, is surprisingly effective and efficient. In addition, when comparing alternative transfection vectors, neocartilage tissue or a cartilage matrix of neocartilage or juvenile cartilage particles, or a combination thereof and a biocompatible chondro-conductive/inductive matrix, is more effectively transfected with certain recombinant vectors.

Preferably, the recombinant vector comprises a promoter sequence operably linked to a polynucleotide encoding a protein, the promoter sequence being capable of driving the expression of said polynucleotide in said neocartilage tissue. Optionally, the polynucleotide may encode a selectable marker protein operably linked to a second promoter sequence so that, e.g., successfully transfected neocartilage can be isolated from untransfected neocatrilage, transfection success rates may be determined, among other observations known to be achievable from using markers with vectors.

The vectors of the present invention used to transfect the neocartilage are preferably an adenovirus, adeno-associated virus or retrovirus. Those of skill in the art will recognize that adeno-associated viruses and retroviruses having enhanced neocartilage binding properties, and higher concomitant chondrocyte cell and neocartilage transduction/transfection efficiencies, may be determined by methods disclosed by Erguang et al. and other methods known in the art. L. Erguang, et al. Integrin αᵥβ₁ is an Adenovirus Coreceptor. J. Virol. (2001) 75(11):5405-5409. Other vectors associated with indirect transfection methods displaying enhanced binding with neocartilage may be determined by methods known to those skilled in the art. Such enhanced vectors may be viruses, lipids or lipid-like delivery vehicles, electrotransfection means and the like, and are meant to be encompassed by the scope of the present invention.

Most preferably, the recombinant vector is adenovirus fiber type 51. As shown in the figures and examples of the present invention, adenovirus fiber type 51 was able to transduce between 10-20% of the total cell population in 45-day-old neocartilage cultures compared with approximately 1% transduction rates of prior methods. The transduction efficiency did not change with 100-day-old neocartilage cultures. Thus, the present invention provides a method for transfecting neocartilage tissue, the neocartilage tissue comprising at least one chondrocyte cell, to confer on at least about 10% of all individual chondrocyte cells the ability to express a desired protein. As noted above, the method comprises infecting the cultured neocartilage tissue with a recombinant vector, the recombinant vector comprising a polynucleotide encoding said protein.

Without being bound by a particular theory, the inventors believe that adenovirus fiber type 51 comprises an enhanced ability to bind with αᵥβ₁ receptors of the neocartilage and/or neocartilage comprises an enhanced ability to bind αᵥβ₁ ligand in vitro (See L. Erguang, et al. Integrin αᵥβ₁ is an Adenovirus Coreceptor. J. Virol. (2001) 75(11):5405-5409). Thus, those viruses and cartilage tissues having enhanced αᵥβ₁ ligand/receptor binding are encompassed within the scope of the present invention.

In another embodiment of the present invention, an isolated chondrocyte cell comprising a recombinant adenovirus vector comprising a polynucleotide encoding a protein operably linked to a promoter sequence, wherein said promoter sequence drives the expression of said polynucleotide in said isolated chondrocyte cell. In addition, a composition of matter is provided comprising intact neocartilage tissue or a cartilage matrix, said intact neocartilage or cartilage matrix comprising a recombinant adenovirus vector further comprising a polynucleotide encoding a protein operably linked to a promoter sequence, wherein said promoter sequence drives the expression of said polynucleotide in said intact neocartilage tissue or cartilage matrix.

These and other embodiments provide an alternative explant model to identify and validate therapeutic targets for extracellular matrix degeneration.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

Transfection of Neocartilage using Adenoviral Vector

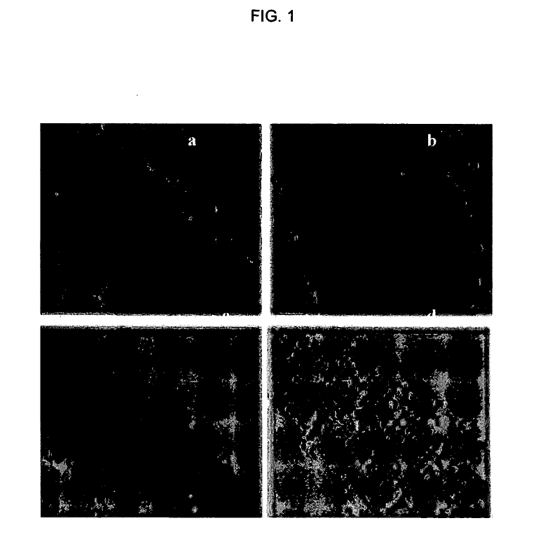

The hyaline-like neocartilage system described above was used for monitoring the biological impact of over-expressed gene constructs, extracellular matrix degradation was measured following transduction with adenovirus expressing ADAM-TS4 and MMP-13. However, those of skill in the art will recognize that the same protocols herein may be performed using well-known modifications using a cartilage matrix of neocartilage or juvenile cartilage particles, or a combination thereof and a biocompatible chondro-conductive/inductive matrix. Expressed ADAM-TS4 resulted in significant loss of proteoglycan as determined by Safarin-O staining (FIG. 1). No loss of proteoglycan was observed in MMP-13 and LacZ controls. DMMB analysis of proteoglycan release confirmed the loss of proteoglycan content by Safarin-O histological evaluation.

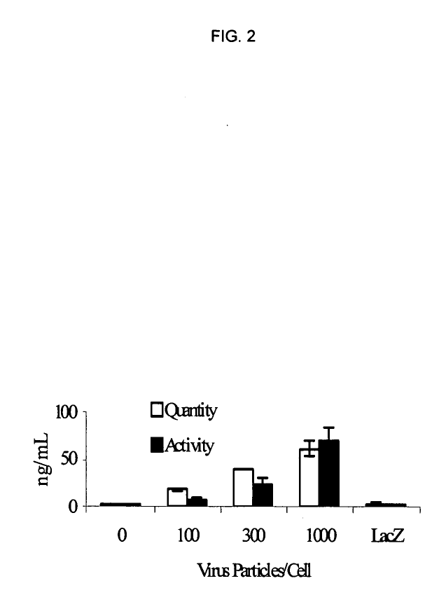

Following adenovirus delivery of constitutively active MMP-13, both protein expression and activity were detected and levels correlate with the concentration (virus particles/cell) of adenovirus delivered as compared to LacZ control at 1000 virus particles per cell (FIG. 2).

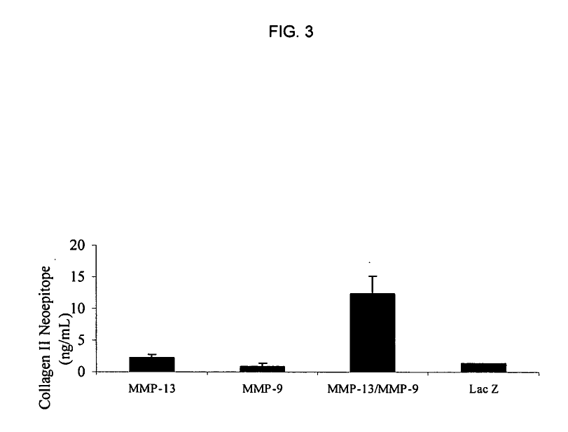

Overexpression of MMP-13 did not induce the release of type II collagen neoepitope as compared to LacZ transduced control (FIG. 3). Furthermore, the addition of 20 units/ml testicular hyaluronidase, used to deplete proteoglycan from the tissue, did not influence the release of MMP-13 generated type II collagen neoepitope.

Therefore, the release of the MMP-13 generated neoepitope was dependent on a gelatinase, such as MMP-9. Following the addition of MMP-9 protein to MMP-13 transduced neocartilage; there was a significant release of type II collagen neoepitope observed (FIG. 3). No significant release of neoepitope was observed in neocartilage transduced with MMP-13 or treated with MMP-9 protein alone.

Unlike articular cartilage, tissue engineered neocartilage can be transduced with adenoviral vectors with sufficient efficiency to measure functional outcomes. Adenovirus mediated ADAM-TS4 gene expression resulted in proteoglycan release from the neocartilage tissue, whereas no proteoglycan release was observed following adenovirus mediated MMP-13 or LacZ expression. No significant release of type II collagen neoepitope was observed following MMP-13 expression. Without being bound to a particular theory, it is believed that increased collagen cross-linking occurs with age in humans (D. R. Eyre et al. J. Biochem. (1988)). This cross-linking may help retain MMP-13 generated neoepitope and the release is dependent on additional enzymatic cleavage such as that from MMP-9. Using the neocartilage system described above, the neocartilage tissue was genetically manipulated using adenovirus constructs to identify the release of MMP-13 generated collagen II neoepitopes. Thus, neocartilage, unlike human articular cartilage, is amenable to adenovirus infection and thus the potential use of it in studying the effects of specific targets on human cartilage degradation.

Tissue Culture—Human neocartilage discs were supplied by Isto Technologies (St. Louis, Mo.) and cultured as previously described in a 96-well format. Donor cartilage tissue was obtained from an accredited Tissue Procurement Organization. The discs comprise chondrocytes which were isolated from the proximal tibia and distal femur bones from children (range, newborn-15 years; mean, 7.5 years±5) and adults (range 18-72 years; mean, 37.3±14.5) within 18 to 24 hours of accidental death (Mid-America Transplant Services, St Louis, Mo.). Chondrocytes derived from human fetal cartilage were isolated similarly from the proximal tibia and distal femur (18 to 22 weeks gestation, Advanced Bioscience Resources, Alameda, Calif.) within 24 hours of death.

Hyaline cartilage was dissected and transferred to HL-1™ Complete serum-free media (BioWhittaker, Walkersville, Md.) to remove contaminating synovial fluid. Next, cartilage was cut into 1-mm cubes, washed, and transferred to 50 mL sterile conical tubes (4 g tissue per tube) for 30-minute digestion in protease from Streptomyces griseus (2 mg/mL, Sigma, St Louis, Mo.), followed by overnight incubation in HL-1™ containing 2000 units CLS2 collagenase (Worthington, Lakewood, N.J.) and 5 mg Type VIII hyaluronidase (Sigma, St Louis, Mo.) at 37° C. with mechanical agitation. The next morning, cell suspensions were diluted with 10 mL of fresh media, vortexed gently, and tissue debris was removed by gravity filtration through 70-μm Falcon cell strainer units (Beckton Dickenson, Franklin Lakes, N.J.). Cells were pelleted at 2000 rpm for 8 minutes and counted to determine viability (>98%) before plating at a density of 1 to 2×10⁵ cells/cm² in 1 mL of Dulbecco's Modified Eagle's Medium containing 10% heat-inactivated fetal bovine serum (Summit Biotechnology, Ft Collins, Colo.). Initial plating of the cells in 10% fetal bovine serum was necessary to achieve chondrocyte adherence to the plastic surface. Cultures were maintained for as many as 300 days under defined serum-free conditions in a 37° C. humidified atmosphere of 95% air, 5% CO₂.

HL-1™ Complete serum-free medium was used to stimulate extracellular matrix synthesis of high-density monolayer chondrocyte cultures. HL-1™ is a chemically defined medium containing less than 30 μg protein per mL. Components of HL-1™ include water for injection, a modified Dulbecco's Modified Eagle's Medium/F12 base, Hepes buffer, known amounts of insulin, transferrin, testosterone, sodium selenite, ethanolamine, various saturated and unsaturated fatty acids, proprietary stabilizing proteins, and β-glycerol phosphate. HL-1™ does not contain bovine serum albumin or other undefined protein mixtures. The following protocol resulted in reproducible tissue formation. The serum content of the culture medium was reduced gradually from 10% on Day 3 to 5% on Day 7 (2 mL total volume) and to less than 2% on Day 10 of culture, after addition of HL-1™ Complete serum-free medium. Ascorbate (50 μg/mL) was added at each media exchange, beginning on Day 3. It was discovered that removal of ⅔ of the total volume of spent media and replacement with fresh HL-1™ provided a conditioned media that enhanced neocartilage formation in chondrocyte cultures derived from prepubescent donors. Unless otherwise indicated, media exchange occurred twice weekly.

Adenovirus—Adenoviruses were generated by Galapagos Genomics (Netherlands). Constitutively active MMP-13 cDNA was generated as previously described (J. M. Freije et al. J. Biol. Chem. (1988)). Wild type cDNAs for ADAM-TS4 (aggrecanase-1) and LacZ were cloned.

Infection of Neocartilage—Neocartilage discs were infected with 0-1000 virus particles per cell overnight in HL-1™ media (Biowhitaker). Culture media was collected every 24 hours. Testicular hyaluronidase and MMP-9 protein were used at 20 units/ml and 5 ng/ml, respectively.

Biochemical Assays—A sandwich ELISA was used to quantify total MMP-13 and activity was determined using R&D Systems (Minneapolis, Minn.) fluorokine enzyme ELISA assay. Type II collagen neoepitope release was assayed using a sandwich ELISA.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method for transfecting neocartilage tissue or a cartilage matrix to confer on said tissue or matrix the ability to express a desired protein, the method comprising infecting cultured neocartilage tissue or a cartilage matrix with a recombinant vector, said recombinant vector comprising a polynucleotide encoding said protein.

2. A method according to claim 1, wherein the recombinant vector further comprises a promoter sequence operably linked to said polynucleotide encoding said protein, said promoter sequence capable of driving the expression of said polynucleotide in said neocartilage tissue or cartilage matrix.

3. A method according to claim 2, wherein said polynucleotide further encodes a selectable marker protein operably linked to a second promoter sequence.

4. A method according to claim 1, wherein the vector is selected from the group consisting of an adenovirus, adeno-associated virus and retrovirus.

5. A method according to claim 4, wherein the adenovirus is adenovirus fiber type 51.

6. A method according to claim 1, wherein said neocartilage tissue or a cartilage matrix is mammalian neocartilage tissue or a mammalian cartilage matrix.

7. A method according to claim 6, wherein said mammalian neocartilage tissue or mammalian cartilage matrix is a human neocartilage tissue or a human cartilage matrix.

8. A method according to claim 1, wherein the cartilage matrix comprises:
a cartilage matrix of neocartilage or juvenile cartilage particles, or a combination thereof; and
a biocompatible chondro-conductive/inductive matrix.

9. A method according to claim 8, wherein the cartilage matrix comprises particles of neocartilage.

10. A method according to claim 8, wherein the cartilage matrix comprises particles of juvenile cartilage.

11. A method according to claim 8, wherein the biocompatible chondro-conductive/inductive matrix is selected from the group consisting of fibrinogen, fibrinogen/thrombin, albumin, in-situ forming PEG hydrogel, fibrin/hyaluronate, fibrin/collagen/hyaluronate, PEG/hyaluronate, PEG/collagen, other plasma and protein-based adhesives and sealants, other natural adhesives and sealants and any combination thereof.

12. A method according to claim 11, wherein the biocompatible chondro-conductive/inductive matrix further comprises an osteo-conductive matrix.

13. A method according to claim 1, wherein at least about 5% of chondrocytes comprising the neocartilage tissue or cartilage matrix are conferred the ability to express the desired protein.

14. A method according to claim 13, wherein at least about 10% of chondrocytes comprising the neocartilage tissue or cartilage matrix are conferred the ability to express the desired protein.

15. An isolated chondrocyte cell comprising a recombinant adenovirus vector comprising a polynucleotide encoding a protein operably linked to a promoter sequence, wherein said promoter sequence drives the expression of said polynucleotide in said isolated chondrocyte cell.

16. A composition of matter comprising intact neocartilage tissue or a cartilage matrix, said intact neocartilage tissue or cartilage matrix comprising a recombinant adenovirus vector further comprising a polynucleotide encoding a protein operably linked to a promoter sequence, wherein said promoter sequence drives the expression of said polynucleotide in said intact neocartilage tissue or cartilage matrix.
